(19) United States
(12) Patent Application Publication
(10) Pub. No.: US 2022/0288999 A1
(43) Pub. Date: Sep. 15, 2022

(54) VEHICLE

(71) Applicant: TOYOTA JIDOSHA KABUSHIKI KAISHA, Toyota-shi (JP)

(72) Inventor: Yamato NIWA, Kariya-shi (JP)

(73) Assignee: TOYOTA JIDOSHA KABUSHIKI KAISHA, Toyota-shi (JP)

(21) Appl. No.: 17/669,582

(22) Filed: Feb. 11, 2022

(30) Foreign Application Priority Data
Mar. 11, 2021 (JP) 2021-039312

(51) Int. Cl. B60H 3/00; B60H 1/00; E05B 77/54

(52) U.S. Cl. CPC B60H 3/0035; B60H 1/00742; B60H 1/00828; E05B 77/54; B60H 2003/0042

(57) ABSTRACT

A vehicle includes a lock device, a gas generator, an air conditioner, and an ECU. The gas generator generates, in a vehicle compartment of the vehicle, a gas having a sterilizing effect or a deodorizing effect. The air conditioner ventilates the vehicle compartment. The ECU starts operation of the gas generator when a request for operation of the gas generator is made and the door is in the locked state, starts ventilation by the air conditioner after the gas generator is stopped, and keeps the door in the locked state when a request to switch from the locked state to the unlocked state is made during a period from start of the operation of the gas generator to end of the ventilation by the air conditioner.

This nonprovisional application is based on Japanese Patent Application No. 2021-039312 filed on Mar. 11, 2021 with the Japan Patent Office, the entire contents of which are hereby incorporated by reference.

### BACKGROUND

### Field

The present disclosure relates to a vehicle, and more particularly, to a vehicle equipped with a gas generator.

### Description of the Background Art

Japanese Patent Laying-Open No. 2007-022186 discloses a vehicle deodorization apparatus including an ozone generator. The vehicle deodorization apparatus deodorizes the inside of the vehicle using ozone. When it is detected that there is no occupant in the vehicle, the vehicle deodorization apparatus performs a strong deodorization operation. When it is detected that the door is opened, the apparatus shifts to the forced exhaust routine.

The gas generator generates a gas having a sterilization effect, such as ozone in the vehicle compartment. Therefore, it is not preferable that the gas remains in the vehicle compartment in a state where the concentration of the gas is high when the user gets on. Even when the forced exhaust routine is executed as described in Japanese Patent Laying-Open No. 2007-022186, there is a possibility that the gas remains in the vehicle compartment in a high concentration state when the user gets on the vehicle immediately after opening the door.

### SUMMARY

The present disclosure has been made in view of the above problem. An object of the present disclosure is to avoid a situation in which the gas remains in the vehicle compartment in a high concentration state when the user gets on the vehicle in which the gas generator that generates the gas having the sterilization effect is mounted.

A vehicle of the present disclosure includes a lock device, a gas generator, an air conditioner, and a control device. The lock device switches a door of the vehicle to either a locked state or an unlocked state. The gas generator generates, in a vehicle compartment of the vehicle, a gas having a sterilizing effect or a deodorizing effect. The air conditioner ventilates the vehicle compartment. The control device controls the lock device, the gas generator, and the air conditioner. The control device: starts operation of the gas generator when a request for operation of the gas generator is made and the door is in the locked state; starts ventilation by the air conditioner after the gas generator is stopped; and keeps the door in the locked state when a request to switch from the locked state to the unlocked state is made during a period from start of the operation of the gas generator to end of the ventilation by the air conditioner.

This configuration prevents a user from opening the door during the period from the start of operation of the gas generator to the end of ventilation by the air conditioner. As a result, the situation where the gas of a high concentration remains in the vehicle compartment when the user rides on the vehicle is avoided.

After the ventilation by the air conditioner, the control device may permit switch from the locked state to the unlocked state.

This configuration limits the period during which the user can ride on the vehicle to the period in which the concentration of the gas is low enough for the user to ride on the vehicle. As a result, the situation where the gas of a high concentration remains in the vehicle compartment when the user rides on the vehicle is avoided.

When the request to switch from the locked state to the unlocked state is made during a period from start of the operation of the gas generator to end of the ventilation by the air conditioner, the control device may inform a user of a time when the switch is to be permitted, or a time interval to the time when the switch is to be permitted. This configuration enables enhancement of the user convenience.

The control device may stop the gas generator when a request to switch from the locked state to the unlocked state is made during operation of the gas generator, and start ventilation by the air conditioner after the gas generator is stopped. This configuration can make the time to permit switch from the locked state to the unlocked state earlier.

The vehicle may further include an occupant detection sensor that detects presence and absence of an occupant in the vehicle compartment. When the occupant detection sensor detects absence of the occupant and the door is in the locked state, the control device may start operation of the gas generator.

This configuration enables reliable confirmation of the absence of an occupant in the vehicle compartment and enables generation of the gas.

The vehicle may further include an occupant detection sensor that detects presence and absence of an occupant in the vehicle compartment. When the occupant detection sensor detects the occupant during operation of the gas generator, the control device may control the lock device to switch the door from the locked state to the unlocked state.

This configuration can prevent the situation where the user is locked in the vehicle compartment in which the gas is being generated.

The vehicle may further include a gas concentration sensor that detects a concentration of the gas in the vehicle compartment. When the concentration of the gas detected by the gas concentration sensor becomes lower than a threshold value during ventilation by the air conditioner, the control device may permit switch from the locked state to the unlocked state.

This configuration can prevent the situation where the user uselessly waits for permission of switch from the locked state to the unlocked state.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a diagram showing an overall configuration of a vehicle according to the present embodiment.

FIG. 2 is a diagram showing main components relating to sterilization in the vehicle according to the present embodiment.

FIG. 3 is a flow chart showing an example of a procedure of processing involved in sterilization of the vehicle compartment in the first embodiment.

FIG. 4 is a timing chart showing enabled state/disable state of a door unlock operation, ON/OFF of a gas generator, ON/OFF of an air circulation function in the vehicle compartment, and ON/OFF of an air ventilation function in the vehicle compartment.

FIG. 5 is a flow chart showing an example of a procedure of processing involved in sterilization of the vehicle compartment in the second embodiment.

FIG. 6 is a flow chart showing an example of a procedure of processing involved in sterilization of the vehicle compartment in the third embodiment.

FIG. 7 is a diagram showing an example of a screen displayed on a display device of a user terminal.

### DESCRIPTION OF THE PREFERRED EMBODIMENTS

Hereinafter, embodiments of the present disclosure will be described in detail with reference to the drawings. In the drawings, the same or corresponding portions are denoted by the same reference numerals, and the description thereof will not be repeated.

In the following embodiments, ozone is mainly used as an example of a gas having a sterilization effect. In other aspects, ions such as silver ions or other gases such as chlorine-based gases may be used instead of ozone.

### Embodiment 1

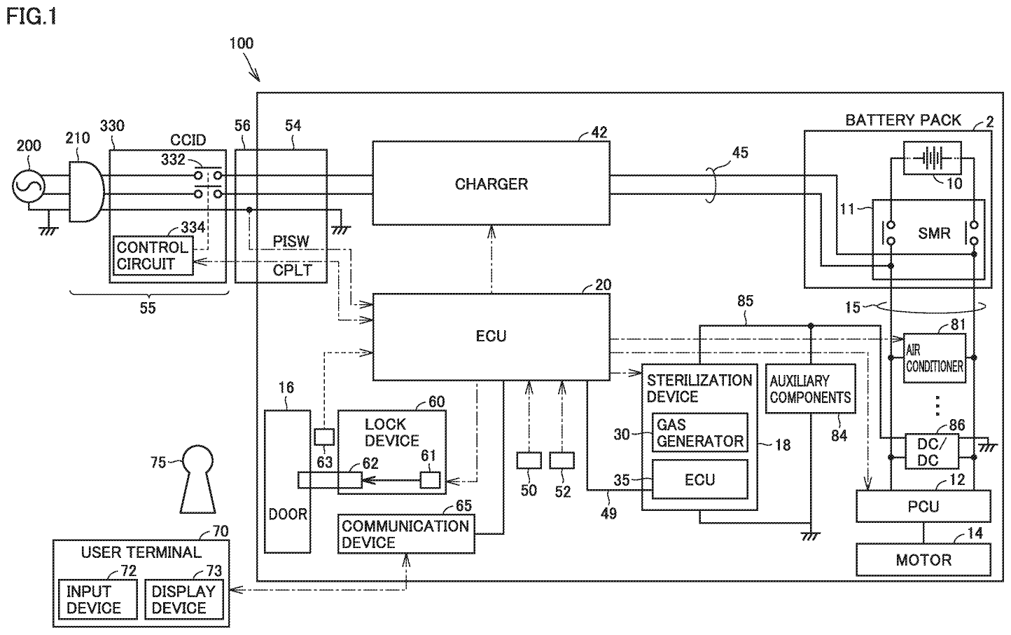

FIG. 1 is a diagram showing an overall configuration of a vehicle according to the present embodiment. The vehicle 100 is an electric vehicle that travels using electric power from a battery. In this embodiment, an example in which the vehicle 100 is an electric vehicle will be described. In another aspect, the vehicle 100 may be a hybrid vehicle further equipped with an engine (not shown). In yet another aspect, the vehicle 100 may be a fuel cell vehicle in which a fuel cell (not shown) is further mounted.

Referring to FIG. 1, vehicle 100 includes battery pack 2, PCU 12, motor 14, ECU (control device) 20, DC/DC converter 86, and auxiliary components 84. The vehicle 100 further includes an inlet 54 and a charger 42, as components for external charging. The vehicle 100 further includes a sterilization device 18, an air conditioner 81, an occupant detection sensor 50, and a gas concentration sensor 52, as components for sterilization in the vehicle compartment. The vehicle 100 includes a door 16, a door opening/closing sensor 63, a lock device 60, and a communication device 65, as components for opening and closing and locking the door.

The battery pack 2 includes a battery 10 and a system main relay (SMR) 11.

The battery 10 is a power storage device configured to be chargeable and dischargeable. The battery 10 is, for example, a lithium ion battery or a secondary battery such as a nickel-hydrogen battery or a lead-acid battery. Instead of the battery 10, a power storage device including a power storage element such as an electric double layer capacitor may be used.

The battery 10 is connected to the PCU 12 via the SMR 11 and the power line 15. When the SMR 11 is on while the vehicle 100 is traveling, power from the battery 10 is supplied to the PCU 12. When the motor 14 generates electric power during braking of the vehicle 100, the generated electric power is supplied to the PCU 12 and then stored in the battery 10.

The PCU 12 includes a converter and an inverter (both not shown). The converter boosts the voltage of the power from the battery 10. The inverter converts DC power supplied from the converter into AC power to drive the motor 14. The PCU 12 is controlled in accordance with a control signal from the ECU 20. The converter need not be provided.

The motor 14 is an AC rotating electrical machine. The motor 14 is, for example, a permanent magnet type synchronous motor including a rotor in which permanent magnets are embedded. The motor 14 drives wheels (not shown) of the vehicle 100 by rotating the motor 14 using electric power supplied from the PCU 12. Thus, the vehicle 100 travels.

The ECU 20 incorporates a CPU (Central Processing Unit) and a memory (none of which are shown). The CPU controls each device of the vehicle 100 in accordance with information or the like stored in the memory. The memory includes a ROM (Read Only Memory) and a RAM (Random Access Memory). The ROM stores programs executed by the CPU. The RAM temporarily stores data referred to by the CPU. Control of the ECU 20 is realized by software processing. The control of the ECU 20 may be possible by hardware fabricated in the ECU 20.

The DC/DC converter 86 is connected between the power line 15 and the power line 85. The DC/DC converter 86 converts the output voltage of the battery 10. The DC/DC converter 86 can output the converted power of the voltage to the power line 85. The power is used for the operation of the sterilization device 18 (described below) and the operation of the auxiliary components 84 connected to the power line 85 in parallel with the sterilization device 18.

The inlet 54 is configured to be connectable to the connector 56 of the charging cable 55. The cable connection signal PISW is output from the connector 56 to the ECU 20. The cable connection signal PISW is used by the ECU 20 to determine whether the connector 56 and the inlet 54 are connected to each other.

The charging cable 55 includes a plug 210 and a CCID (Charging Circuit Interrupt Device) box 330. The plug 210 is connected to a power source 200 external to the vehicle 100. The CCID box 330 includes a relay 332 and a control circuit 334.

When the plug 210 is connected to the power source 200 and the connector 56 is connected to the inlet 54, the ECU 20 and the control circuit 334 can exchange various information through the control pilot signal CPLT. This information includes information indicating the on/off state of the relay 332, the magnitude of the charging current, and the like.

The ECU 20 outputs a request to the control circuit 334 to close the relay 332, for example, through the control pilot signal CPLT. The control circuit 334 closes the relay 332 in response to the request. The control circuit 334 transmits information indicating that the relay 332 is closed to the ECU 20 via the control pilot signal CPLT. Thus, the ECU 20 can start charging the vehicle 100.

The input end of the charger 42 is connected to the inlet 54. The output terminal of the charger 42 is connected to the battery pack 2. When the power source 200 and the inlet 54 are connected to each other, upon receiving a charge start command from the ECU 20, the charger 42 converts AC power from the power source 200 into charging power (DC power) for the battery 10. Specifically, the charger 42 converts the voltage of the received power into a DC voltage suitable for charging the battery 10. The charger 42 outputs the converted power of the voltage to the power line 45.

The sterilization device 18 generates gas for sterilizing the vehicle compartment of the vehicle 100. The vehicle compartment is a space in which an occupant rides in the vehicle 100. The sterilization device 18 is controlled in accordance with a control signal from the ECU 20. The sterilization device 18 operates using electric power output from the DC/DC converter 86. The sterilization device 18 includes a gas generator 30 and an ECU 35. The gas generator 30 is provided in a vehicle compartment. The gas generator 30 generates ozone having a sterilization effect, in the vehicle compartment. The ECU 35 controls the gas generator 30. The ECU 35 is connected to the ECU 20 via a local bus 49.

The air conditioner 81 operates in accordance with a control command from the ECU 20. Thus, the air conditioner 81 circulates air in the vehicle compartment (circulation function) and ventilates air in the vehicle compartment (ventilation function). The air conditioner 81 circulates ozone in the passenger compartment after the gas generator 30 generates ozone, for example. Accordingly, ozone is filled in the vehicle compartment, so that the vehicle compartment is more efficiently sterilized. Thereafter, the air conditioner 81 ventilates the air in the vehicle compartment. As a result, it is possible to avoid a situation where ozone remains in the vehicle compartment in a state where the concentration of ozone is high after ventilation.

The occupant detection sensor 50 detects whether or not an occupant is in the passenger compartment of the vehicle 100. The occupant detection sensor 50 is, for example, a pressure sensor, and detects the presence or absence of occupant detection according to whether or not the pressure in the seat of the vehicle 100 is equal to or higher than a threshold value. The detection result is output to the ECU 20.

The gas concentration sensor 52 detects the concentration of ozone generated from the gas generator 30 in the vehicle compartment. The detected concentration is output to the ECU 20.

The door 16 separates the inside and the outside of the vehicle compartment of the vehicle 100. The door opening/closing sensor 63 is configured to detect whether the door 16 is open or closed. The door opening/closing sensor 63 is, for example, a limit switch, a proximity sensor, or a photoelectric sensor. The detection result of the door opening/closing sensor 63 is output to the ECU 20.

The lock device 60 switches the door 16 between a locked state and an unlocked state in accordance with an instruction from the ECU 20. The lock device 60 is configured to be able to switch the door 16 from the unlocked state to the locked state, for example, when the lock device 60 receives a command to switch the door 16 from the unlocked state to the locked state (a door lock command), from the ECU 20. On the other hand, the lock device 60 is configured to switch the door 16 from the locked state to the unlocked state when the lock device 60 receives a command to switch the door 16 from the locked state to the unlocked state (door unlock command), from the ECU 20.

The lock device 60 includes a door lock mechanism 62 and an actuator 61. The door lock mechanism 62 is, for example, an engagement member (e.g., a pin or a pawl) configured to be capable of switching between an engaged state and a disengaged state with respect to the door 16 in a closed state.

The actuator 61 is configured to switch the door lock mechanism 62 from one of the engaged state and the disengaged state with respect to the door 16 to the other. When the door lock mechanism 62 is switched between the engaged state and the disengaged state, the door 16 is switched between the door locked state and the door unlocked state.

The communication device 65 is an interface for the ECU 20 to wirelessly communicate with an electronic key (not shown) of the user 75 or a user terminal 70 (e.g., a smartphone, tablet, or wearable device). The communication device 65 is connected to the ECU 20. The communication device 65 transmits information from the ECU 20 to the electronic key or the user terminal 70. The communication device 65 transmits information received from the electronic key or the user terminal 70 to the ECU 20.

The user 75 operates the electronic key. Thus, the user 75 can switch the state of the door 16 from one of the door lock state and the door unlock state to the other.

For example, when the user 75 operates the lock button of the electronic key when the door 16 is closed and in the unlocked state, a signal indicating that the lock button has been operated is transmitted from the electronic key to the ECU 20 via the communication device 65. Upon receipt of the signal, the ECU 20 transmits a lock command to the lock device 60. Thus, the door 16 is switched from the door unlocked state to the door locked state.

On the other hand, when the user 75 operates the unlock button of the electronic key when the door 16 is closed and in the locked state, a signal indicating that the unlock button has been operated is transmitted from the electronic key to the ECU 20 via the communication device 65. Upon receipt of the signal, the ECU 20 transmits an unlock command to the lock device 60. Thus, the state of the door 16 is switched from the door lock state to the door unlock state.

Hereinafter, a user operation for requesting switching from the door unlock state to the door lock state is also referred to as a “door lock operation”. A user operation for switching from the door lock state to the door unlock state is also referred to as a “door unlock operation”.

The ECU 20 determines whether the door 16 is in the locked state or the unlocked state according to the state of the actuator 61.

When the user 75 operates a sterilization start button (not shown) of the input device 72 of the user terminal 70, the sterilization of the vehicle compartment is started. Specifically, when the button is operated, the user terminal 70 outputs a signal indicating a trigger to start sterilization (that is, a trigger to request the gas generator 30 to operate). The ECU 20 receives the signal via the communication device 65. Thereafter, the ECU 20 starts the gas generator 30. Thus, the sterilization of the vehicle compartment starts with the start of the generation of ozone. When the sterilization start button is provided in the vehicle compartment, the ECU 20 may be configured to activate the gas generator 30 after a predetermined time elapses from the operation time of the button when the button is operated by the user in the vehicle compartment.

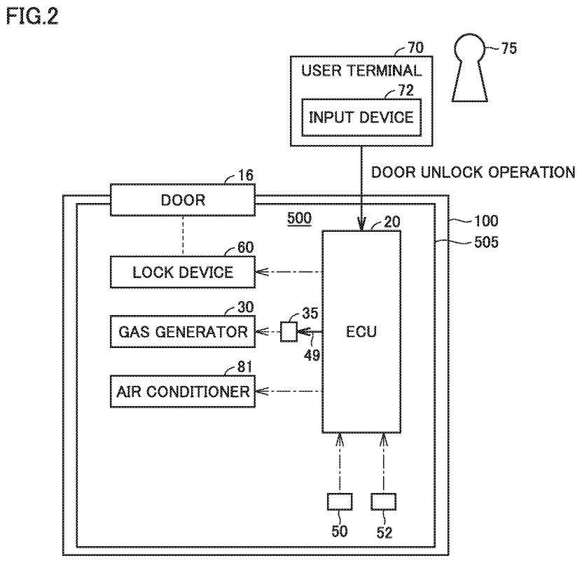

FIG. 2 is a diagram showing main components relating to sterilization in the vehicle 100 according to the present embodiment. In the following description, for simplicity of description, the ECU 20 controls the gas generator 30 through the ECU 35 connected via the local bus 49. The ECU 20 functions as a “control device” for controlling the lock device 60, the gas generator 30, and the air conditioner 81.

When the gas generator 30 generates ozone in the vehicle compartment, it is not preferable that the ozone remains in the vehicle compartment 505 at a high concentration when the user 75 gets on the vehicle after the generation of the ozone. Thus, in the vehicle 100 according to the present embodiment, the ECU 20 maintains the door 16 in the locked state when switching from the locked state to the unlocked state is requested (i.e., the door unlock operation is performed) during a period from the operation start time of the gas generator 30 (the time when the gas generator 30 starts to generate ozone) to the end (completion) time of ventilation by the air conditioner 81. In this case, the fact that the lock state of the door 16 is maintained without being permitted by the ECU 20 to switch from the door lock state to the door unlock state, is described as “the door unlock operation is disable”.

Since the door unlock operation is disabled, the state of the door 16 is not switched from the door lock state to the door unlock state by the operation at least during the above-described period. Therefore, a situation in which the user 75 cannot open the door 16 during the above-described period is achieved. The ECU 20 enables the door unlock operation after the period has elapsed (that is, after the time when ventilation in the vehicle compartment 505 is completed). The enablement of the door unlock operation means that switching from the door lock state to the door unlock state is permitted by the ECU 20 when the door unlock operation is performed.

When the door unlock operation is enabled, the user 75 can open the door 16 by the operation after the door unlock operation is enabled. When the user 75 gets on, ventilation in the vehicle compartment 505 has already been completed. As a result, a situation in which ozone remains in the vehicle compartment 505 in a high concentration state is avoided. An embodiment in which the ECU 20 uses the output from the occupant detection sensor 50 or the gas concentration sensor 52 will be described later.

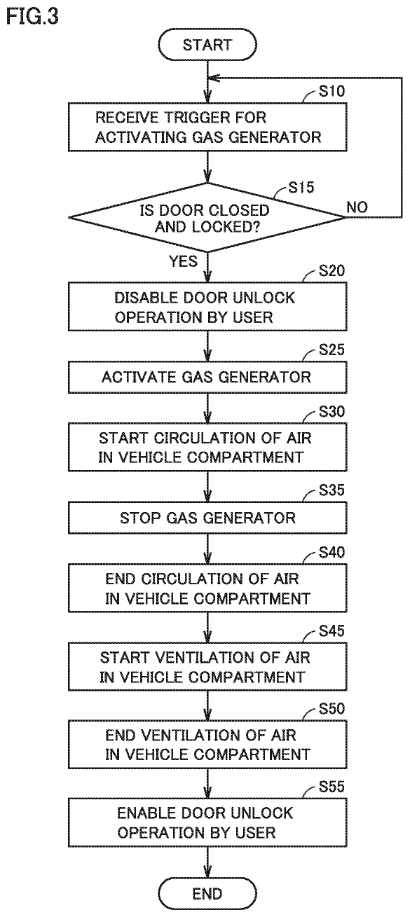

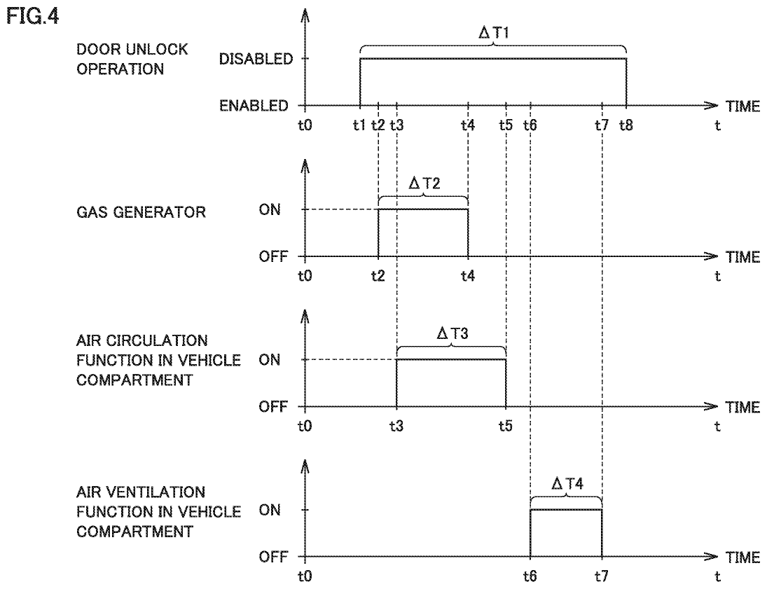

FIG. 3 is a flow chart showing an example of the procedure of processing involving sterilization in the vehicle compartment 505, in the first embodiment. FIG. 4 is a timing chart showing enabled state/disabled state of the door unlock operation, ON/OFF of the gas generator 30, ON/OFF of the circulation function of the air in the vehicle compartment 505, and ON/OFF of the ventilation function of the air in the vehicle compartment 505. Hereinafter, FIG. 4 will be referred to as appropriate in the description of FIG. 3.

Referring to FIG. 3, ECU 20 receives a trigger for starting (activating) gas generator 30 (step S10). The trigger is, for example, a signal output from the user terminal 70 to the ECU 20 when the user 75 operates the sterilization start button. Upon receipt of the trigger (that is, when the operation of the gas generator 30 is requested), the ECU 20 proceeds to step S15.

In step S15, ECU 20 determines whether door 16 is closed and locked. The ECU 20 performs the determination based on, for example, the output of the door opening/closing sensor 63 and the state of the actuator 61. If the door 16 is closed and locked (YES in step S15), the ECU 20 proceeds to step S20 to start sterilization in the vehicle compartment 505. Otherwise (NO in step S15), ECU 20 returns the process to step S10.

At time t1 in FIG. 4, the ECU 20 disables the door unlock operation by the user 75 (step S20). The disablement of the operation is maintained until time t8 (described later) at which the process of step S55 is performed (that is, during the period ΔT1). As a result, the user 75 cannot get on the vehicle between when the gas generator 30 generates ozone and when the ozone is sufficiently discharged to the outside of the vehicle compartment.

At time t2, ECU 20 activates gas generator 30 (step S25). Thus, ozone starts to be generated from the gas generator 30. The generation of ozone continues until the period ΔT2 elapses from time t2. The period ΔT2 is appropriately determined in advance so that the amount of ozone required for sterilization in the vehicle compartment 505 is generated at the time t4 when the generation of ozone is finished. The processes of step S20 and step S25 may be performed simultaneously. That is, the time t1 at which the door unlock operation starts to be disabled and the time t2 at which the generation of ozone starts may be the same.

The ECU 20 outputs a command for starting circulation of air in the vehicle compartment 505 to the air conditioner 81 (step S30). Thus, the circulation of the air containing ozone in the vehicle compartment 505 starts at time t3. As a result, the inside of the vehicle compartment 505 is effectively sterilized. The circulation of air by the air conditioner 81 is continued until the period ΔT3 elapses from time t3. The period ΔT3 is appropriately determined in advance so that the ozone in the vehicle compartment 505 is sufficiently circulated (filled) at the time t5 when the circulation of the air is completed. The processes of step S25 and step S30 may be performed simultaneously. That is, the time t2 at which the generation of ozone is started and the time t3 at which the circulation of air is started may be the same.

Next, the ECU 20 stops the gas generator 30 at time t4 when the period ΔT2 has elapsed from time t2 (step S35).

Next, the ECU 20 ends the circulation of the air in the vehicle compartment 505 by the air conditioner 81 at time t5 when the period ΔT3 has elapsed from time t3 (step S40).

Next, the ECU 20 starts ventilation of the air in the vehicle compartment 505 by the air conditioner 81 at time t6 (step S45). Thus, the ozone sufficiently circulated (filled) in the vehicle compartment 505 begins to be discharged to the outside of the vehicle. Ventilation by the air conditioner 81 is continued until a period ΔT4 has elapsed from time t6. The period ΔT4 is appropriately determined so as to be a period sufficient for the concentration of ozone in the vehicle compartment 505 to fall below the threshold value at time t7 of the end of ventilation. The threshold value is a concentration when the concentration of ozone in the vehicle compartment 505 is low so that the passenger may get on the vehicle, and is appropriately determined. The processes of step S40 and step S45 may be performed simultaneously. That is, the time t5 of the end of circulation of air and the time t6 of the start of ventilation may be the same.

Next, the ECU 20 ends ventilation by the air conditioner 81 at time t7 when the period ΔT4 has elapsed from time t6 (step S50).

Upon completion of ventilation, the ECU 20 enables the door unlock operation by the user 75 at time t8 (step S55). In the period after the time t8 at which the operation is enabled, unlike the period ΔT1 during which the operation is disabled, when the operation is performed, the door 16 is unlocked (that is, the unlocked state is switched to the locked state). Therefore, the user can rid the vehicle by opening the door 16 after the operation. The processes of step S50 and step S55 may be performed simultaneously. That is, the time t7 at which ventilation ends and the time t8 at which the door unlock operation is enabled may be the same. After step S55, ECU 20 ends the series of processes.

As described above, in the first embodiment, the door unlock operation by the user 75 is disabled during the period ΔT1 including the period from the start of operation of the gas generator 30 (time t2) to the end of ventilation by the air conditioner 81 (time t7). Specifically, even when the ECU 20 receives the door unlock operation from the user 75 during the period ΔT1, the ECU 20 keeps the door 16 in the locked state without switching the door 16 from the locked state to the unlocked state.

Then, after the end of ventilation, the door unlock operation is enabled (switching from the locked state to the unlocked state is permitted). Therefore, the user 75 can rid the vehicle by opening the door 16 after the door unlock operation. The time at which the user 75 can get on the vehicle (that is, the time after the time t8) is the time after the time t7. At time t7, the concentration of ozone in the vehicle compartment 505 is less than the threshold value as ventilation in the vehicle compartment 505 is finished.

Therefore, according to the first embodiment, even when the inside of the vehicle compartment 505 is sterilized by using ozone, a situation in which ozone remains in the vehicle compartment 505 at a high concentration when the user 75 gets on the vehicle, is avoided. In particular, there is a case where the inside of the vehicle compartment 505 is sterilized after a large number of people indefinite in car sharing or the like get on the vehicle 100. The present embodiment is useful for another user who rides on the vehicle 100 after such sterilization.

### Embodiment 2

In the second embodiment, the ECU 20 determines whether or not to start the operation of the gas generator 30 in accordance with the detection result of the occupant detection sensor 50. In the following description, FIG. 4 will be referred to as appropriate.

In the second embodiment, the ECU 20 starts the operation of the gas generator 30 when a following condition is further satisfied before the time t1 (FIG. 4) at which the door unlock operation is disabled. This condition is that the occupant detection sensor 50 detects the absence of the occupant in the vehicle compartment 505.

The configuration of the vehicle in the second embodiment is the same as that of the vehicle 100 (FIGS. 1 and 2) in the first embodiment.

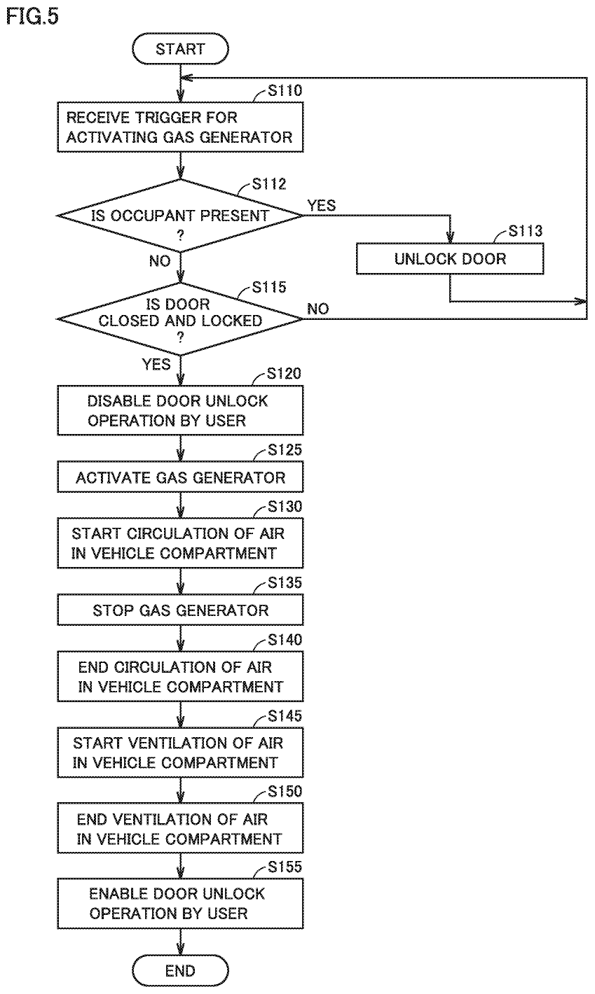

FIG. 5 is a flowchart showing an example of a procedure of a process involving sterilization in the vehicle compartment 505, according to the second embodiment. The processes of steps S10 and S155 to S155 in FIG. 5 are the same as the processes of steps S10 and S15 to S55 in FIG. 3, respectively.

Referring to FIG. 5, ECU 20 receives a trigger for starting gas generator 30 (step S110). Then, the ECU 20 determines whether or not there is an occupant in the vehicle compartment 505 in accordance with the detection result of the occupant detection sensor 50 (step S112).

If it is determined in step S112 that there is an occupant in the vehicle compartment 505 (YES in step S112), the ECU 20 controls the lock device 60 to unlock the door 16 (i.e., switch from the locked state to the unlocked state) (step S113). This allows the occupant to exit the vehicle compartment 505 before the generation of ozone. After the door 16 is opened, the process returns to step S110.

On the other hand, if it is determined in step S112 that the occupant is not in the vehicle compartment 505 (NO in step S112), the ECU 20 proceeds to step S115.

If it is determined in step S115 that the door 16 is closed and locked (YES in step S115), the ECU 20 disables the door unlock operation by the user 75 (step S120). Then, the ECU 20 starts the gas generator 30 (step S125). The subsequent processes of steps S130 to S155 are the same as the processes of steps S30 to S55 in FIG. 3, respectively.

As described above, in the second embodiment, the ECU 20 starts the operation of the gas generator 30 when the occupant detection sensor 50 detects the absence of the occupant (YES in step S112) and the door 16 is in the locked state (YES in step S115). Thus, it is possible to reliably check the absence of an occupant in the vehicle compartment 505 before the gas generator 30 starts to operate. As a result, it is possible to avoid a situation where ozone begins to be generated in a situation where an occupant is in the vehicle compartment 505.

### Modification 1 of Embodiment 2

In the second embodiment, a unit including a camera and an image processing circuit (none of which are shown) may be used as the occupant detection sensor 50 instead of the pressure sensor. In this case, the image processing circuit performs image processing on an image captured by the camera. As a result, whether or not an occupant is in the vehicle compartment 505 is detected. Alternatively, a proximity sensor or the like may be used as the occupant detection sensor 50.

### Modification 2 of Embodiment 2

In the second embodiment and the first modified example thereof, when the absence of an occupant in the vehicle compartment 505 is detected before the period ΔT1 (FIG. 4) during which the door unlock operation is disabled (before step S120 in FIG. 5), the ECU 20 starts the operation of the gas generator 30. On the other hand, when the occupant detection sensor 50 detects the occupant during the period ΔT1 during which the door unlock operation is disabled (from step S120 to step S150 in FIG. 5), the ECU 20 may control the lock device 60 to immediately unlock the door 16. Thus, the door 16 can be opened immediately without confining the user 75 in the vehicle compartment 505 while ozone is being generated (filled) in the vehicle compartment 505. As a result, even after time t2 at which ozone is generated, the occupant can immediately exit the vehicle compartment 505.

### Embodiment 3

In the third embodiment, the ECU 20 determines whether or not to terminate ventilation in the vehicle compartment 505 according to the detection value of the gas concentration sensor 52. In the following description, FIG. 4 will be referred to as appropriate.

Specifically, when the gas concentration detected by the gas concentration sensor 52 falls below the threshold value (That is, the concentration when the concentration of ozone in the vehicle compartment 505 is low so that the passenger may get on the vehicle), during ventilation by the air conditioner 81, the ECU 20 ends ventilation by the air conditioner 81. Then, the ECU 20 permits switching of the door 16 from the locked state to the unlocked state (that is, enables the door unlock operation).

The overall configuration of the vehicle in the third embodiment is the same as that of the vehicle 100 (FIGS. 1 and 2) in the first embodiment.

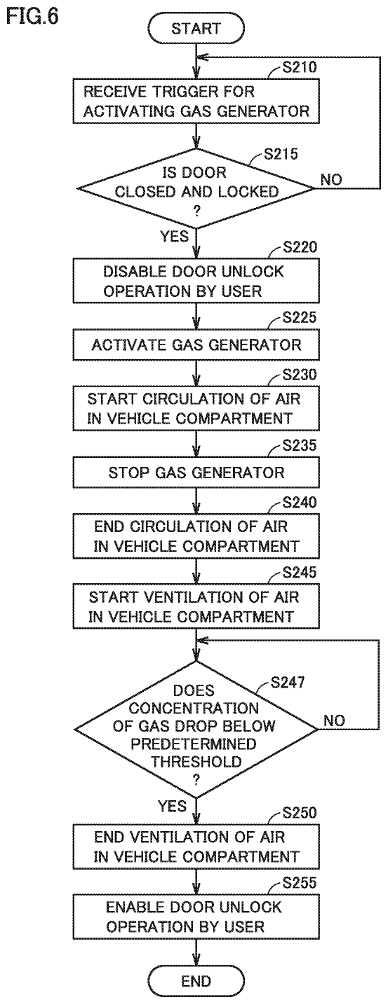

FIG. 6 is a flow chart showing an example of the procedure of processing involving sterilization in the vehicle compartment 505, in the third embodiment. Referring to FIG. 6, the processes of steps S210 to S245, S250 and S255 are the same as the processes of steps S10 to S45, S50 and S55 of FIG. 3, respectively. Hereinafter, FIG. 4 will be referred to as appropriate in the description of FIG. 6.

When ventilation by the air conditioner 81 is started at time t6 (FIG. 4) (step S245), in step S247, the ECU 20 determines whether or not the concentration of ozone in the vehicle compartment 505 has dropped below the threshold value according to the detection value of the gas concentration sensor 52. The determination process is performed at predetermined time intervals.

In step S247, when the concentration of ozone is equal to or higher than the threshold value (NO in step S247), the ECU 20 continues ventilation by the air conditioner 81 until the concentration of ozone drops below the threshold value (until the branch of YES is followed in step S247).

On the other hand, if it is determined in step S247 that the concentration of ozone has dropped below the threshold value (YES in step S247), the ECU 20 stops the air conditioner 81 to terminate ventilation in the vehicle compartment 505 (step S250). Thereafter, in step S255, the door unlock operation is enabled. Thereafter, the ECU 20 ends the series of processes.

As described above, in the third embodiment, when the gas concentration detected by the gas concentration sensor 52 falls below the threshold value during ventilation by the air conditioner 81, the ECU 20 terminates ventilation and enables the door unlock operation (that is, allows switching from the locked state to the unlocked state).

As a result, unlike the case where the length of the ventilation period ΔT4 is predetermined (first embodiment), the door lock state is not maintained unnecessarily due to the fact that the predetermined period ΔT4 has not elapsed even though the concentration of ozone in the vehicle compartment 505 actually drops below the threshold value. Thus, it is possible to avoid a situation where the user unnecessarily waits until the time when the door unlock operation is enabled.

### Embodiment 4

In the fourth embodiment, when the ECU 20 receives the door unlock operation during a period in which the door unlock operation is disabled (corresponding to the period ΔT1 in FIG. 4), the user 75 is notified in advance. This notification notifies the user 75 of the time interval to the time when the door unlock operation is enabled.

The overall configuration of the vehicle in the fourth embodiment is the same as that of the vehicle 100 (FIGS. 1 and 2) in the first embodiment.

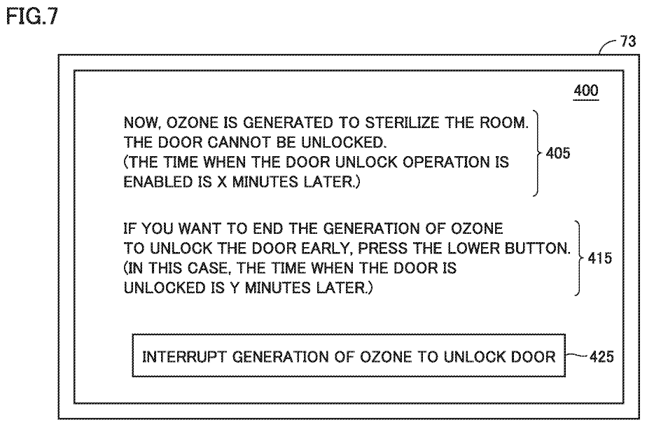

FIG. 7 is a diagram showing an example of a screen displayed on the display device 73 of the user terminal 70. In the following description, FIGS. 3 and 4 will be referred to as appropriate.

When the door unlocking operation is performed during the period ΔT1 (FIG. 4) during which the door unlocking operation is disabled (that is, during the period from step S20 to step S50 in FIG. 3), the ECU 20 performs a notification to the user 75. This notification notifies the user of the time interval to the time when the door unlock operation is enabled. For example, the ECU 20 outputs a signal indicating the time when the door unlock operation is enabled, to the user terminal 70 via the communication device 65. The user terminal 70 displays a screen 400 indicating the time on the display device 73, in accordance with the signal.

Screen 400 includes message 405 and 415 and button 425. The message 405 indicates to the user 75 that the door unlock operation is disabled at the time (current time) when the screen 400 is displayed. The message 405 displays the time interval (X in the example of FIG. 7) from the current time to the time t8 (FIG. 4) at which the door unlock operation is to be enabled to the user 75. Accordingly, the user 75 can know in advance the time at which the user 75 can enters the vehicle compartment 505 in association with the enablement of the door unlock operation.

The value of X is calculated by the ECU 20, for example, in accordance with the length of the predetermined period ΔT1 (FIG. 4) or in accordance with the detection value of the gas concentration sensor 52. For example, when the value of X is calculated in accordance with the length of the period ΔT1, the ECU 20 calculates the value of X by subtracting the time interval between the time t1 (the time when the door unlock operation starts to be disabled) and the current time from the length of the predetermined period ΔT1. When the value of X is calculated in accordance with the detection value of the gas concentration sensor 52, the ECU 20 calculates the value of X by predicting the time at which the detection value falls to the aforementioned threshold value, based on the detection value and the history of the temporal change of the detection value.

Thus, the user 75 is notified of the time interval to the time when the door unlock operation is enabled. As a result, the user 75 can know the time in advance.

While the gas generator 30 is operating (That is, the period from the time t2 (FIG. 4) at which the generation of ozone is started to the time t4 at which the generation of ozone is scheduled to be ended), the user 75 knows the time interval (X minutes) indicated in the message 405. The user 75 may wish to get on the vehicle earlier than the time X minutes after the current time.

Therefore, the message 415 indicates that the time interval to the time when the door unlock operation is enabled can be changed when the button 425 is operated by the user 75 during the above-described period. Specifically, the message 415 indicates that the time interval to the time when the door unlock operation is enabled can be changed to a time interval (Y minutes) earlier than the time interval (X minutes) displayed in the message 405 (Y<X).

The button 425 is provided to allow the user 75 to get on the vehicle earlier even if the generation of ozone by the gas generator 30 is interrupted. Specifically, when the button 425 is operated during the operation of the gas generator 30, the user terminal 70 outputs a request to the ECU 20 to stop the gas generator 30. Further, the user terminal 70 outputs a request to the ECU 20 so that the vehicle compartment is ventilated by the air conditioner 81 after the gas generator 30 is stopped. The user terminal 70 further outputs a request to the ECU 20 to enable the unlock operation after the end of ventilation by the air conditioner 81. These requests are transmitted to the ECU 20 via the communication device 65. In response to these requests, the ECU 20 stops the gas generator 30 before time t4 at which the generation of ozone is scheduled to be terminated (e.g., when the request is received). Next, the ECU 20 enables the unlock operation after completion of ventilation by the air conditioner 81.

Accordingly, the time when the gas generator 30 stops becomes earlier than the time t4 scheduled when the button 425 is not operated. Therefore, when the button 425 is operated during the operation of the gas generator 30, the period during which ozone is generated from the gas generator 30 is shorter than the period ΔT2 when the button 425 is not operated. Therefore, when the button 425 is operated, the total amount of ozone generated from the gas generator 30 is smaller than the total amount when the button 425 is not pressed.

Accordingly, the period required for circulating air in the vehicle compartment 505 and the period required for ventilation in the vehicle compartment 505 can be set shorter than the periods ΔT3 and ΔT4 in the first embodiment. Therefore, when the button 425 is operated, the end time of ventilation can be made earlier than the time t7 when the button 425 is not operated. Thus, the time when the door unlock operation is enabled after the time t7 when the button 425 is not operated can be made earlier than the time t8 when the button 425 is not operated. As a result, the demand of the user who desires to use the vehicle in an early stage can be met.

The value of Y is determined in accordance with the length of a period from the time t2 when the operation of the gas generator 30 is started to the time when the button 425 is operated (time when the generation of ozone is interrupted). For example, depending on the length of the period, the start time and the end time (corresponding to the time t3 and the time t5, respectively) of the period ΔT3 of circulation of air in the vehicle compartment 505 and the start time and the end time (corresponding to the time t6 and the time t7, respectively) of the period ΔT4 of ventilation are appropriately set to be earlier. In accordance with these times, the ECU 20 determines the time at which the door unlock operation is enabled (corresponding to the time t8). Then, the ECU 20 determines the value of Y according to this time and the current time.

In the screen 400, the user 75 is notified of the time interval (X minutes or Y minutes) to the time at which the switching from the locked state to the unlocked state is permitted, but the user 75 may be notified of the time instead of the time interval to the time.

As described above, in the fourth embodiment, when switching of the door 16 from the locked state to the unlocked state is requested while the door unlock operation is disabled, the ECU 20 notifies the user 75 of the time at which the switching is permitted or the time interval to the time. Thus, the convenience of the user 75 can be improved.

Further, the user 75 can select whether to wait until the time at which the door unlock operation is enabled (time X minutes after the current time) or to get on the vehicle at a time earlier than the current time (time Y minutes after the current time). When the user 75 waits until the time X minutes after the current time, the inside of the vehicle compartment 505 is sufficiently sterilized. When the user 75 gets on the vehicle 100 at a time Y minutes after the current time, the user 75 can use the vehicle 100 as soon as possible. As a result, user convenience can be further improved.

[Other Variations]

In the fourth embodiment, the time interval to the time when the door unlock operation is enabled (for example, X minutes or Y minutes) may be notified to the user 75 in advance by voice via the microphone (not shown) of the user terminal 70. This makes it possible to improve the convenience of a user who does not have sufficient vision.

In the first to fourth embodiments described above, the gas generator 30 generates gas mainly having a sterilization effect. On the other hand, the present disclosure can also be applied to an embodiment in which the gas generator 30 generates a gas having a deodorizing effect. In this case, by appropriately replacing “sterilization” with “deodorization” in the above embodiments, the effect of the present disclosure can be similarly achieved.

Although the vehicle 100 capable of external charging has been described in the first to fourth embodiments, the vehicle of the present disclosure is not limited to the vehicle capable of external charging. Specifically, the inlet 54 and the charger 42 in FIG. 1 do not necessarily have to be provided in the vehicle 100.

The ECU 20 may change the time t3 (FIG. 4) at which the circulation of air starts so as to be closer to the time t2 at which the generation of ozone starts (for example, so that the time t3 becomes equal to the time t2). Thus, the time at which the air containing ozone is sufficiently circulated (filled) in the vehicle compartment 505 can be made earlier than the time t5. As a result, the ventilation start time after the circulation of the air, the ventilation end time after the period ΔT4 from this time, and the time when the door unlock operation is enabled after the ventilation end time can be made earlier than the time t6, the time t7, and the time t8 in FIG. 4, respectively. Accordingly, the time at which the user 75 can get on the vehicle can be made earlier than the time t8. As a result, the convenience of the user 75 can be further improved.

Although the present disclosure has been described and illustrated in detail, it is clearly understood that the same is by way of illustration and example only and is not to be taken by way of limitation, the scope of the present disclosure being interpreted by the terms of the appended claims.

What is claimed is:

1. A vehicle comprising:
a lock device that switches a door of the vehicle to either a locked state or an unlocked state;
a gas generator that generates, in a vehicle compartment of the vehicle, a gas having a sterilizing effect or a deodorizing effect;
an air conditioner that ventilates the vehicle compartment; and
a control device that controls the lock device, the gas generator, and the air conditioner, wherein
the control device
starts operation of the gas generator when a request for operation of the gas generator is made and the door is in the locked state,
starts ventilation by the air conditioner after the gas generator is stopped, and
keeps the door in the locked state when a request to switch from the locked state to the unlocked state is made during a period from start of the operation of the gas generator to end of the ventilation by the air conditioner.

2. The vehicle according to claim 1, wherein after the ventilation by the air conditioner, the control device permits switch from the locked state to the unlocked state.

3. The vehicle according to claim 2, wherein, when the request to switch from the locked state to the unlocked state is made during a period from start of the operation of the gas generator to end of the ventilation by the air conditioner, the control device informs a user of a time when the switch is to be permitted, or a time interval to the time when the switch is to be permitted.

4. The vehicle according to claim 1, wherein
the control device
stops the gas generator when a request to switch from the locked state to the unlocked state is made during operation of the gas generator, and
starts ventilation by the air conditioner after the gas generator is stopped.

5. The vehicle according to claim 1, further comprising an occupant detection sensor that detects presence and absence of an occupant in the vehicle compartment, wherein
when the occupant detection sensor detects absence of the occupant and the door is in the locked state, the control device starts operation of the gas generator.

6. The vehicle according to claim 1, further comprising an occupant detection sensor that detects presence and absence of an occupant in the vehicle compartment, wherein
when the occupant detection sensor detects the occupant during operation of the gas generator, the control device controls the lock device to switch the door from the locked state to the unlocked state.

7. The vehicle according to claim 1, further comprising a gas concentration sensor that detects a concentration of the gas in the vehicle compartment, wherein
when the concentration of the gas detected by the gas concentration sensor becomes lower than a threshold value during ventilation by the air conditioner, the control device permits switch from the locked state to the unlocked state.
